(19) United States
(12) Patent Application Publication
(10) Pub. No.: US 2013/0324814 A1
(43) Pub. Date: Dec. 5, 2013

(54) ESTIMATION OF SYSTEMIC VASCULAR RESISTANCE AND CARDIAC OUTPUT USING ARTERIAL PULSE OXIMETRY WAVEFORMS

(71) Applicant: Albert Maarek, Miami, FL (US)

(72) Inventor: Albert Maarek, Miami, FL (US)

(21) Appl. No.: 13/991,694

(22) PCT Filed: Dec. 6, 2011
(86) PCT No.: PCT/IB11/02935

§ 371 Date: Aug. 14, 2013

(30) Foreign Application Priority Data
Dec. 6, 2010 (IB) PCT/IB2010/003114
Nov. 18, 2011 (IB) 1119982.5

(51) Int. Cl. A61B 5/02; A61B 5/1455; A61B 5/00; A61B 5/024

(52) U.S. Cl. CPC A61B 5/02028; A61B 5/02416; A61B 5/14551; A61B 5/7257
USPC 600/324

(57) ABSTRACT

The invention relates to a method to estimate sytemic vascular resistance (SVR) and cardiac output (CO) from a pulse oximeter (photoplethysmography, PTG), comprising the steps of generating arterial blood pulse wave forms having first and second peaks, generating a Fast Fourier Transform and calculating the ratio of the height of the second peak (B) to the first peak (A) across the entire measurement using the equation FFTRI to estimate SVR, and the equation CO=(SR/FFTRI)×80 to estimate CO, where SR=S2/S1 and wherein SI and S2 are the areas under the whole PTG wave and the part in diastolic phase, respectively.

$${\sum }_{n = 2}^{N} {FFT}^{2} ( {f}_{n} ) / {\sum }_{n = 1}^{N} {FFT}^{2} ( {f}_{n} ) .$$

The present application relates to an apparatus and method to measure cardiac output. More specifically the invention relates to a method of measuring cardiac output using a pulse oximeter and use of a pulse oximeter to measure cardiac output.

Cardiovascular disease (CVD) is the number one cause of death globally and is projected to remain the leading cause of death (WHO, 2008). An estimated 17.5 million people died from cardiovascular disease in 2005, representing 30% of all global deaths. Of these deaths, 7.6 million were due to heart attacks and 5.7 million were due to stroke. Many Americans are suffering from the spectrum of coronary artery disease, CVD, and diabetes (Sanders, 2003; WES Oxiburger, 2002), three of the most prevalent chronic diseases. More than 50 million Americans (>30% of the population) have hypertension, which results in inordinate medical expenses (TNHBPEPCC, 2003), and more than 700,000 Americans lose their life to heart disease every year, the leading cause of death in the United States (CDC, 2005; USDHHS, 2004).

It is well known that heart rate, blood pressure, arterial stiffness and cardiac output (CO) are all essential physiological parameters of the human cardiovascular system.

CO, defined as the blood volume ejected by the heart per minute in L/min, is regarded as the ultimate expression of cardiovascular performance, since CO indicates how well the heart is able to provide enough nutrition and oxygen to the peripheral organs and tissues. For human beings, in order to maintain a normal state of tissue perfusion and oxygen delivery condition, the baseline CO should be in the range of 4-8 L/min. If CO gets out of this range, it is often a sign of CVD, such as hypertension, stroke, or heart failure. Hence, regular CO monitoring plays an essential role in the evaluation, treatment, and follow-up of critically ill patients. Accordingly, a non-invasive, inexpensive, safe, and fast device that can assess CO and other hemodynamic parameters would be a suitable alternative to other techniques that are invasive, expensive, and risky in CVD patients.

Ideally, a technology which measures CO should be non-invasive, accurate, and reliable. At present, no single method meets all these criteria.

Intermittent thermo-dilution is widely accepted as the clinical golden standard. This method requires the insertion of a pulmonary artery catheter to obtain one measurement every 3-4 minutes (Mathews & Singh, 2008). However, this procedure is too invasive.

Two existing less invasive and continuous methods are esophageal Doppler monitoring and CO₂ re-breathing, but both of these require skilled operators and expensive measurement devices (Mathews & Singh, 2008).

Among the currently used methods, impedance cardiography is probably the only non-invasive and automatic technique. However, the impedance device is big and expensive, and its accuracy is often influenced by the change of electrode positions and the sweat on the skin (Richard et al., 2001).

Due to the disadvantages mentioned above, these methods are all unquestionably limited to bedside use. They are not portable or wearable, so they are difficult to incorporate into home health care monitoring systems as well.

To solve this problem, one of the best ways is to derive a new CO indicator from photoplethysmography (PPG).

According to the present invention there is provides a method to estimate Systemic Vascular Resistance from arterial blood waveform analysis, the method comprising the steps of generating pulse wave forms each having first and second peaks using a pulse oximeter over a time period, generating a Fast Fourier Transform to calculate the ratio of the height of the second peak to the first peak across the entire measurement using the formula

$$FFT RI = {\sum }_{n = 2}^{N} {FFT}^{2} ( {f}_{n} ) / {\sum }_{n = 1}^{N} {FFT}^{2} ( {f}_{n} ) .$$

to estimate Reflection Index and thus Systemic Vascular Resistance (SVR).

The invention further provides a method to estimate Cardiac Output (CO) from a waveform generated over time by a pulse oximeter using the equation

CO=(SR/FFTRI)×80

wherein FFTRI is as calculated above and and

SR=S2/S1 wherein S1 and S2 are the areas under the whole PTG wave and the part of wave in diastolic phase.

The time period of generation of the waveform is between 30 seconds and 5 minutes. More preferably the time period is from 1 to 3 minutes.

The waveform can be generated from a pulse oximeter reading from arterial blood.

The reading may be generated at a home computer and sent via email to a practitioner.

The invention further relates to an apparatus and method to estimate cardiac output average during a 2 minute time period using an oximeter to provide the heart rate detection and arterial blood waveform.

The invention is described herein with reference to the following figures wherein

FIG. 1 shows the original waveform (PTG) generated by an oximeter

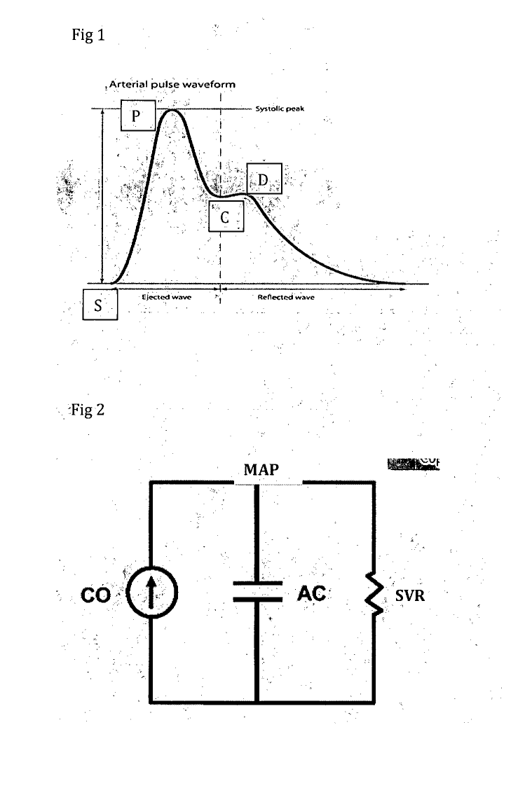

FIG. 2 shows the two-element Windkessel model

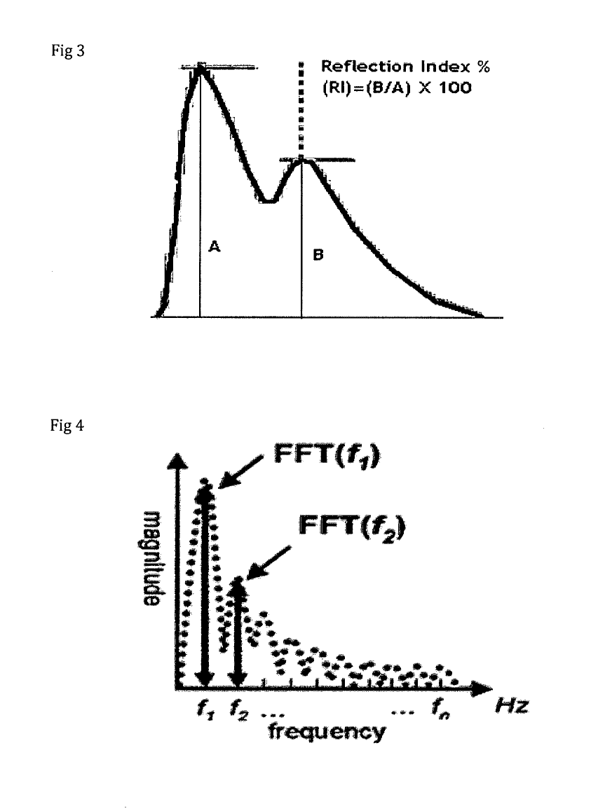

FIG. 3 shows the Reflection Index calculation

FIG. 4 is the PTG wave in the frequency domain. FFT (f)

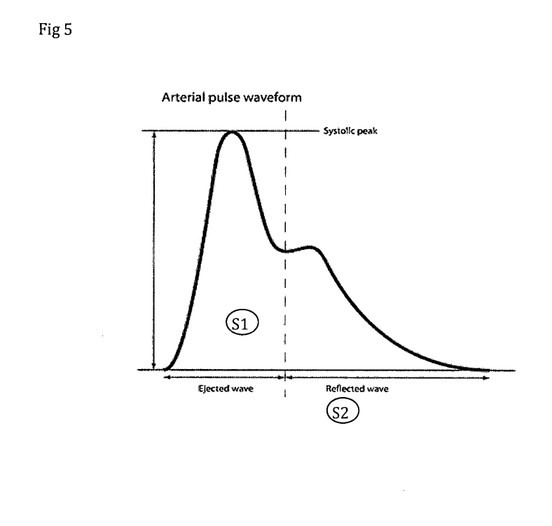

FIG. 5 is the PTG wave in the time domain.

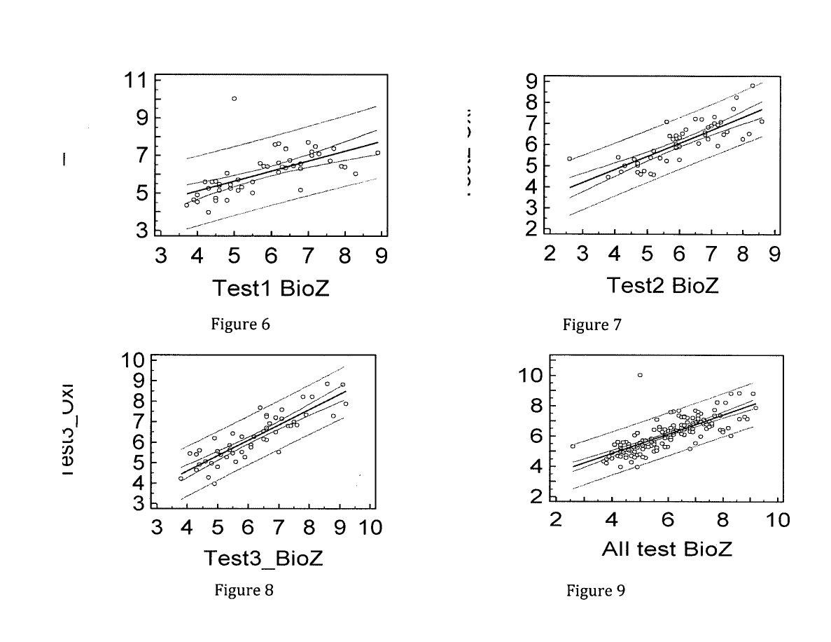

FIG. 6 shows the correlation between CO measured using ESO and BioZ Dx at baseline

FIG. 7 shows the correlation between CO measured using ESO and BioZ Dx after first exercise stage

FIG. 8 shows the correlation between CO measured using ESO and BioZ Dx after second exercise stage

FIG. 9 shows the correlation between CO measured using ESO and BioZ Dx for all measurements

The cardiac output estimation requires the heart rate variability analysis and the arterial blood waveform analysis.

Calibration with the heart rate variability analysis:

One uses a calibration with the SDNN parameter of the Heart rate variability (HRV) analysis. The SDNN should calibrate with the Cardiac index (CI), then, the cardiac output (CO) is calculated with the formula: CO=CI/BSA (BSA=Body surface area)

Arterial blood waveform analysis. The original waveform (PTG) generated by an oximeter is shown in FIG. 1 wherein

S=Starting point, P=Percussion point, C=Incisura and D=Dicrotic wave

According to the two-element Windkessel model shown in FIG. 2, the cardiovascular system is analogous to a current source connected with a two-element circuit MAP=Mean arterial pressure and SVR=Systemic vascular resistance.

CO can be calculated according to the following formula:

CO=(MAP/SVR)×80 (1)

SVR is firstly initialized by a pair of calibration CO and MAP data, and its value of the current beat in continuous mode is calculated from MAP and estimated CO of the previous beat, iteratively. The main shortcoming of such technique is that it needs either an invasive arterial catheter or a bedside blood pressure device for acquiring the continuous blood pressure measurement.

However, as shown in equation (1), blood pressure measurement is not a necessity for obtaining CO, if proper surrogates of MAP and SVR can be derived from other signals. According to pulse wave analysis, arterial blood pulse could be divided into two waves (i.e. FIG. 1): first peak (point P) and second peak (Point D). From the current knowledge, as shown in the FIG. 3, the ratio Reflection Index % (RI) is calculated from the height of the point D (B in FIG. 3) divided by the height of the point S (A in the FIG. 3), and RI represents the small artery resistance which is the main component of SVR.

The invention use the fast Fourier transform (FFT) in the entire records of wave during 2 minutes time, to determine the ratio Height B and the Height A (FIG. 4). Herein this ratio is called: FFTRI with the formula as follows:

$$FFT RI = {\sum }_{n = 2}^{N} {FFT}^{2} ( {f}_{n} ) / {\sum }_{n = 1}^{N} {FFT}^{2} ( {f}_{n} ) . \qquad ( 2 )$$

FIG. 4 is PTG wave in the frequency domain. FFT (f)

The ratio of surface (SR) of whole PTG wave and diastolic phase surface (FIG. 5) with the formula as follows:

SR=S2/S1 (3)

is related to pulse wave reflection and is strongly correlated with systolic and diastolic blood pressure

FIG. 5 shows the PTG wave in the time domain. S1 and S2 are the areas under the whole PTG wave and the part of wave in diastolic phase

Therefore, FFTRI (corresponding to the SVR) divided by SR (corresponding to MAP), is proposed as a potential CO indicator and expressed as follows:

CO=(SR/FFTRI)×80 (4)

The ability of the formula (4), after calibration with SDNN parameter of the HRV analysis to estimate CO average was evaluated in clinical investigation (Miami University) comparing our results to the BioZ Dx (Thoracic impedance technique).

### Study.

### Estimation of the Cardiac Output (CO or Q)

The Electro Sensor Oxi device uses the photoelectrical plethysmography from a digital oximeter for in a completely non-invasive and fast format to assess CO. The

ESO is a device being utilized in other applications (SpO2% and Heart rate variability analysis), but this is the first assessment comparing CO to a standardized device (the BioZ Dx Diagnostic System). Thus, this type of study is likely to be significantly different from what is typically provided to patients who need their CO checked. The ESO device that will be used in the study will provide another option for assessing CO and other indicators of cardiovascular system function. The purpose of this study is a cross-sectional comparison of the ESO and the BioZ Dx Diagnostic System on CO. The ESO device has never been compared to standardized technology on CO, so we cannot hypothesize specific outcomes for this study, but rather we are executing a formative, pilot study to determine if hypotheses can be generated for future studies.

### Methods

Study Participants. All participants (N=51) were recruited by referrals at the University of Miami Miller School of Medicine campus during 2010. The study was conducted with the approval of the Institutional Review Board for human subjects' research, and participants signed informed consent before commencing in the study. The sample comprised of 49% males (n=25) and 51% females (n=26) with a mean age of 31.1 years (SD=10.8; R=18, 65).

Study Design. Potential participants 18 years of age and over were identified as those who expressed an interest in having their CO assessed. Subjects were not enrolled in the study if they: (1) were unable to consent to the study; (2) were undergoing external defibrillation; (3) had erratic, accelerated, or mechanically-controlled irregular heart rhythms; (4) had arterial fibrillation/flutter; (5) had atrioventricular block; (6) had dyes recently introduced into the bloodstream, such as methylene blue, indocyanine green, indigo carmine, and fluorescein; (7) had significant levels of dysfunctional hemoglogins, such as carboxyhemoglogin or methhemoglobin, which will affect the accuracy of the saturation of peripheral oxygen (SpO2) measurement from the oximeter; (8) had any condition restricting blood flow, such as use of a blood pressure cuff or an extreme in systemic vascular resistance, which could have caused an invalid pulse or SpO₂ reading; (9) used fingernail polish or false fingernails during the testing, which could have caused inaccurate SpO2 readings;

Outcomes and Assessments. Criteria used to select the study assessments included: (1) appropriateness for the population; (2) ease of administration and scoring; (3) the investigators' experience administering these measures; and (4) employment of measures involving a multi-method (i.e., self-report and physical measures) approach to enhance the validity of the overall assessment. Each participant completed a basic demographics and medical history questionnaire. Subjects were assessed with the ESO and BioZ Dx devices in the Medical Wellness Center/Center for Complementary and Integrative Medicine. The entire assessment took less than 1 hour for each participant. Each subject began the assessment by sitting at rest for 5 minutes and then had CO measured simultaneously by the ESO and the BioZ Dx devices. Then, the subject walked for 6 minutes on a treadmill at low to moderate intensity (less than 70% of maximal age-adjusted predicted heart rate [220-age]), and CO was measured simultaneously immediately afterward by the ESO and the BioZ Dx devices. Finally, each subject walked for a second 6-minute bout of exercise on the treadmill at the same intensity, and CO was measured simultaneously immediately afterward by the ESO and the BioZ Dx devices. Thus, 3 measurements were completed: 1 at rest and 2 following 6-minute bouts of low-to-moderate intensity exercise on a treadmill. Subjects were compensated $25 for their participation in the study at the conclusion of all three assessments.

Data Analysis. Data were analyzed using SPSS 18 (SPSS Inc., Chicago, Ill.) for Windows. Frequency and descriptive statistics were calculated on all variables. We used Pearson product-moment correlation to estimate the relationship between ESO and the BioZ Dx CO assessments, while controlling for the BioZ Dx signal strength. We used α=0.05 as the criterion for statistical significance.

### Results

Clinical Measurements. Mean height was 67.9 inches (SD=4.5, R=60, 76), mean weight was 172.7 pounds (SD=42.1, R=118, 300), and mean BMI was 26.4 kg/m² (SD=6.6, R=19.6, 56.7) for the sample.

CO Results. Controlling for BioZ Dx signal strength, the correlations between ESO and the BioZ Dx on CO was r=0.693 (p<0.001) at baseline (FIG. 6), r=0.79 (p<0.001) after the first exercise stage (FIG. 7), and r=0.86 (p<0.0001) after the second exercise stage FIG. 8), respectively. The correlations for all the measurements (153 measurements) between ESO and BioZ Dx on CO was r=0.78 (p<0.0001). (FIG. 9)

### CONCLUSIONS

The results of the study suggest a high correlation between the ESO and the BioZ Dx on CO both at rest and after 2 bouts of low-to-moderate exercise. All subjects completed the assessment without reporting any adverse event, and the assessments were completed in a timely fashion.

### REFERENCES

Centers for Disease Control and Prevention. (2005). Deaths: Leading causes for 2002. Atlanta, Ga.: U.S. Department of Health and Human Services, Centers for Disease Control and Prevention; Report No: 53.

Chowienczyk, P, Kelly, R, MacCallum, H, et al. (1999). Photoplethysmographic assessment of pulse wave reflection: Blunted response to endothelium-dependent beta2-adrenergic vasodilation in type II diabetes mellitus. J Am Coll Cardiol, 34, 2007-2014.

Lax, H, Feinberg, A, & Cohen, B. (1956). Studies of the arterial pulse wave and its modification in the presence of human arteriosclerosis. J Chronic Dis, 3, 618-631.

Mathews, L, & Singh, K. (2008). Cardiac output monitoring. Annals of Cardiac Anesthesia, 11, 56-68.

McCombie, D, RES Oxiner, A, & Asada, H. (2005). Identification of vascular dynamics and estimation of the cardiac output waveform from wearable PPG sensors.

Proceedings of the 27th IEEE International Engineering in Medicine and Biology Conference, EMBC, Shanghai, China, 3490-3493.

Richard, R, Lonsdorfer-Wolf, E, Charloux, A, et al. (2001). Non-invasive cardiac output evaluation during a maximal progressive exercise test, using a new impedance cardiograph device. Eur J Appl Physiol, 85, 202-207.

The National High Blood Pressure Education Program Coordinating Committee (2003) The seventh report of the joint national committee on prevention, detection, evaluation, and treatment of high blood pressure: The JNC 7 report. JAMA, 289, 2560-2572.

United States Department of Health and Human Services. (2004). The health of the United States, 2003. Bethesda, Md.: U.S. Department of Health and Human Services. Wang, L, & Zhang, Y. (2008). A novel photoplethysmogram index for total peripheral resistance after bicycle exercise. Proceedings of the 5th International Conference on Ubiquitous Healthcare, Pusan, Korea, 175-176.

World Health Organization. (2008). Cardiovascular diseases. Available at: http://www.who.int/cardiovascular_diseases/en/index.html. Accessed Mar. 1, 2008.

1. A method to estimate Systemic Vascular Resistance from arterial blood waveform analysis, the method comprising the steps of generating pulse wave foams each having first and second peaks using a pulse oximeter over a time period, generating a Fast Fourier Transform and calculating the ratio of the height of the second peak to the first peak across the entire measurement using the formula
$$FFT RI = {\sum }_{n = 2}^{N} {FFT}^{2} ( {f}_{n} ) / {\sum }_{n = 1}^{N} {FFT}^{2} ( {f}_{n} ) .$$
to estimate Reflection Index % and thus Systemic Vascular Resistance (SVR).

2. A method to estimate Cardiac Output (CO) from a wave form generated over time by a pulse oximeter using the equation
CO=(SR/FFTRI)×80
wherein FFTRI is as calculated in claim 1 and
SR=S2/S1 wherein SI and S2 are the areas under the whole PTG wave and the part of wave in diastolic phase.

3. A method as claimed in claim 1 wherein the time period of generation of the waveform is between 1 and 3 minutes.

4. Use of a pulse oximeter capable of generating arterial blood waveform in a method as claimed in claim 1.

5. Use of a pulse oximeter as claimed in claim 4 in combination with means to remotely send waveform information for CO analysis to a physician.

6. An apparatus to estimate cardiac output average during 2 minute time using an oximeter which can provide heart rate detection and arterial blood wave form for use in the method as set out in claim 2 to estimate and/or monitor CO.
